(19) United States
(12) Patent Application Publication
(10) Pub. No.: US 2026/0157883 A1
(43) Pub. Date: Jun. 11, 2026

(54) Control of Intraocular Pressure During Phacoemulsification

(71) Applicant: JOHNSON & JOHNSON SURGICAL VISION, INC., Irvine, CA (US)

(72) Inventors: Vadim Gliner, Haifa (IL); Alon Boumendil, Haifa (IL); Omry Kadosh, Haifa (IL)

(21) Appl. No.: 18/970,699

(22) Filed: Dec. 5, 2024

(51) Int. Cl. A61F 9/007

(52) U.S. Cl. CPC A61F 9/00736; A61F 9/00781

(57) ABSTRACT

A system, including: a pump, a handpiece including a needle and a sleeve, an irrigation channel forming a fluid path within the handpiece; first and second sensors configured to sense pressure at separate locations along the fluid path; and circuitry configured to: receive first and second pressure measurement signals from the first and second sensors, respectively; identify one or more pressure fluctuations in the first signal and/or the second signal; compare the first signal and the second signal to determine one or more frequency dispersion indicators; characterize the one or more pressure fluctuations based on the one or more frequency dispersion indicators as being associated with an intraocular pressure (IOP) change or a system component; and for a pressure fluctuation associated with the IOP change: determine an estimate of IOP from the first signal; and control the pump based on the estimate of IOP.

### TECHNOLOGICAL FIELD

The present application relates to controlled operation of a phacoemulsification system, and more specifically to phacoemulsification whilst maintaining intraocular pressure.

### BACKGROUND

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then, a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. After removing the cataract with phacoemulsification, the softer outer lens cortex is removed with suction. An intraocular lens is then introduced into the empty lens capsule restoring the patient's vision.

During the phacoemulsification procedure, the eye is irrigated with irrigation fluid (e.g. a balanced salt solution) to maintain intraocular pressure (IOP) within safe limits. Fluid lost from the eye includes fluid removed from the eye by aspiration (denoted herein as aspiration flow) and fluid leaking from the eye through the incision or incisions made at the surgical site.

US20220192878A1 discloses “A phacoemulsification system includes a phacoemulsification probe, an irrigation pump, an aspiration pump, circuitry, and a processor. The probe includes a piezoelectric crystal configured to vibrate at a mechanical resonance frequency of the crystal; a needle configured for insertion into an eye and be vibrated by the crystal to emulsify a lens of the eye; an irrigation channel for receiving irrigation fluid from an irrigation line and flowing the irrigation fluid into the lens capsule; and an aspiration channel for removing material from the lens capsule and evacuating the removed material to an aspiration line. The irrigation and aspiration pumps are coupled with the irrigation and the aspiration line, respectively. The circuitry is configured to measure electrical impedance of the crystal. The processor is configured to receive the measured electrical impedance, and to control an operation of the irrigation pump and the aspiration pump according to the measured electrical impedance.”

### OVERVIEW

A broad aspect of some examples of the disclosure relates to measuring and/or controlling intraocular pressure (IOP) during eye treatment(s) e.g., cataract removal, for example, without directly measuring pressure within the eye.

An aspect of some examples of the disclosure relates to acquiring at least two pressure measurement signals from different locations along a flow path within a channel fluidly connected to a sleeve having a smaller cross sectional flow area than the channel where the sleeve is fluidly connected to the inside of the eye (e.g., fluid within the sleeve experiencing IOP). By comparing the two pressure measurement signals, pressure fluctuations may be characterized as associated with IOP or as pressure changes occurring elsewhere in the system.

In some examples, pressure fluctuations are identified in the pressure measurement signals, e.g., a pressure fluctuation above a threshold occurring at a same time frame for both of the pressure measurement signals.

In some examples, for identified pressure fluctuations, the signals are compared to provide, for each identified pressure fluctuation, one or more indicator(s) of frequency dispersion between the first and second signals. The indicator(s) of frequency dispersion may be compared with a threshold (e.g., each indicator with a corresponding threshold) and, based on the one or more comparison, the identified pressure fluctuation may be characterized as associated with IOP or as associated with the system.

Without wanting to be bound by theory, it is theorized that frequency dispersion occurs to IOP fluctuation signals passing through the small cross sectional flow area of the sleeve. It is theorized that the smaller cross sectional flow area of the sleeve (e.g., associated with surface tension at edges of the flow within the sleeve) disperses changes in IOP where different frequency components of the change in IOP travel at different speeds, providing different pressure signals at different locations (e.g., distances away from the eye) within the channel. It is theorized, that frequency changes occurring elsewhere in the system, e.g., within the irrigation channel, for example, associated with the comparatively large cross sectional flow area of the irrigation channel (e.g., with respect to wavelength of pressure variation and/or with respect to friction at channel walls), will not exhibit (or will exhibit low) dispersion along the irrigation channel (e.g., as measured in the first and second signals).

In some examples, the one or more measures of frequency dispersion between the measurement signals for a specific pressure fluctuation include one or more of a correlation between the signals, an assessment of differential factor between the signals, and a time lag between the signals.

Without wanting to be bound by any theory, it is theorized the two measurements of pressure for a pressure fluctuation associated with system components (and not IOP) will be highly correlated, and, in contrast, a signal exhibiting dispersion will be less corelated. In some examples, the correlation is compared with a threshold and those frequency fluctuations which have correlation above the threshold are characterized as being associated with IOP.

Without wanting to be bound by any theory, it is theorized that an IOP fluctuation in pressure will produce a pressure fluctuation at the proximal sensor which is delayed (having a time lag) compared to the when the pressure fluctuation is measured at the distal sensor. In some examples, the lag (e.g., determined using cross-correlation) is compared to a threshold and those frequency fluctuations which have lag exceeding the threshold are characterized as being associated with IOP.

In some examples, the one or more indicator of frequency dispersion may be determined using a phase differential factor. Where the phase differential factor may then be evaluated to provide one or more parameters characterizing the frequency fluctuation. The parameter(s) may include one or more of a differential factor slope, a weighted average of the phase difference, and a phase difference ratio.

In some examples, the one or more indicator of frequency dispersion may include a comparison of frequency-specific lags, where, if higher frequency bands exhibit larger lags than lower frequency bands the frequency fluctuation may be characterized as associated with IOP.

In some examples, for those frequency fluctuations identified as associated with IOP, IOP may be estimated (e.g., using distal pressure sensor measurement values) and controlled using the estimate. Where a control signal for control of system fluid supply(s) (e.g., control of a pump supplying fluid to the channel) may be generated using the estimated IOP. In some examples, the IOP is compared with a desired IOP (e.g., as specified by a physician) to provide an error signal which may be used as an input to a control architecture which outputs a control signal.

Identified change(s) in IOP may be outputted e.g., communicated to a user and/or saved to a memory and/or the identified change(s) in IOP may be used in generation and/or used to adjust pump control signal(s). In some examples, the one or more measures of frequency dispersion is used as a direct control input to the pump, e.g., where the differential factor is used to generate control signals.

In some examples, the channel is an irrigation channel of a system (e.g., a phacoemulsification system). The phacoemulsification system may include an aspiration channel configured to extract material from the eye, e.g., from a lens capsule. Flow of fluid through the irrigation channel may be controlled to maintain IOP within a desired range while material is being extracted from the eye through the aspiration channel. The aspiration channel may include a phacoemulsification needle where, in some examples, the needle is disposed within a lumen of the sleeve; the outer walls of the needle and inner walls of the sleeve delineating therebetween the cross-sectional flow area of the sleeve.

A potential benefit of using two pressure measurement signals to identify pressure fluctuations as associated with IOP, e.g., as opposed to using a single pressure measurement signal, is increased accuracy of identification of IOP pressure changes potentially enabling more accurate and/or rapid control of IOP.

In the following detailed description, numerous specific details are set forth in order to provide a thorough understanding. However, it will be understood by those skilled in the art that the presently disclosed subject matter may be practiced without these specific details. In other instances, well-known methods and features have not been described in detail so as not to obscure the presently disclosed subject matter.

Unless specifically stated otherwise, as apparent from the following discussions, it is appreciated that throughout the specification discussions utilizing terms such as “processing”, “computing”, “comparing”, or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities.

The various illustrative logical blocks, modules, and/or algorithm steps described in connection with the examples disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing any departure from the scope of the disclosure.

It will also be understood that a system according to the present disclosure may be, at least partly, implemented on a suitably programmed computer. Likewise, the present disclosure contemplates a computer program being readable by a computer for executing the method of the present disclosure. The present disclosure further contemplates a non-transitory computer-readable memory tangibly embodying a program of instructions executable by the computer for executing the method of the present disclosure.

### BRIEF DESCRIPTION OF THE DRAWINGS

In order to better understand the subject matter that is disclosed herein and to exemplify how it may be carried out in practice, examples will now be described, by way of non-limiting example only, with reference to the accompanying drawings, in which:

FIG. 1 is a schematic, pictorial view of an exemplary phacoemulsification system, according to examples of the present disclosure;

FIG. 2 is a simplified schematic cross sectional view of exemplary details of a phacoemulsification system, according to examples of the present disclosure;

FIG. 3 is a simplified plot of fluid pressure, with time, at different locations within a phacoemulsification system using a bench top method for simulating surgical events, according to examples of the present disclosure;

FIG. 4 is a method of controlling a phacoemulsification system, according to examples of the present disclosure; and

FIG. 5 is a plot of pressure measurement signals, with time, according to examples of the present disclosure.

### DETAILED DESCRIPTION

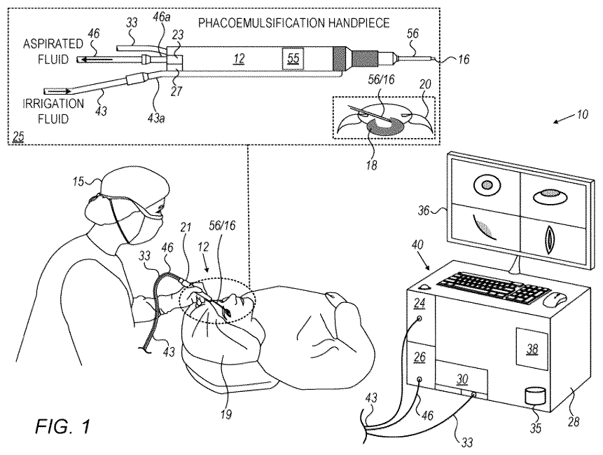

FIG. 1 is a schematic, pictorial view of an exemplary phacoemulsification system 10, according to examples of the present disclosure.

As seen in the pictorial view of phacoemulsification system 10, and in inset 25, phacoemulsification probe 12 (also referred to herein as a handpiece 12) comprises a needle 16 substantially surrounded by an irrigation sleeve 56. Needle 16 is hollow and its lumen is used as an aspiration channel.

Needle 16 is configured for insertion by a physician 15 into an eye 20 of a patient 19, for example into a lens capsule 18 of the eye, to remove a cataract. The needle 16 (and irrigation sleeve 56) are shown in inset 25 as a straight object. However, any suitable needle may be used with phacoemulsification probe 12, for example, a curved or bent tip needle commercially available from Johnson & Johnson Surgical Vision, Inc., Irvine, CA., USA.

Referring now to the irrigation and aspiration modules, irrigation channels 43a and aspiration channel 46a are coupled with irrigation tube 43 and aspiration tube 46, respectively.

Referring now to the system circuitry, and more specifically to sensing circuitry, in the shown example, probe 12 includes a sensor 27 coupled with irrigation channel 43a, and a sensor 23 coupled with aspiration channel 46a. It is noted that other examples may include only one sensor in either of the channels. Generally, one or more sensors may be located at any suitable position along the handpiece, and/or operably attached to the handpiece, such as at a module coupled with or attached at a proximal end of the handpiece.

Sensors 23 and 27 may be any sensor known in the art, including, but not limited to a vacuum sensor or flow sensor. The sensor measurements (e.g., pressure, vacuum, and/or flow) may be taken close to the distal end of the handpiece where the irrigation outlet and the aspiration inlet are located, so as to provide processor 38 an indication of the actual measurements occurring within an eye and provide a short response time to a control loop comprised in processor 38. It is noted that other types of sensors such as temperature sensors, optical sensors, capacitance sensors and/or other sensors may also be incorporated as part of the circuitry of the system.

In some examples, system 10 comprises a processor-controlled irrigation pump 24. During the phacoemulsification procedure, processor-controlled pump 24 comprised in a console 28 pumps irrigation fluid from an irrigation reservoir or tank (not shown) to irrigate the eye via irrigation sleeve 56. The fluid is pumped via irrigation tubing line 43 running from console 28 to probe 12. Irrigation pump 24 may be any pump known in the art, for example, a peristaltic pump or a progressive cavity pump. In some examples, a gravity fed irrigation source such as a balanced salt solution bottle or bag may be used with pump 24 or in replacement of the pump.

In some examples, processor 38 controls a pump rate of irrigation pump 24, for example to maintain IOP within prespecified limits, to enable or disable irrigation, to increase or decrease a flow volume of irrigation fluid, to open or close valves along the irrigation line, or otherwise control irrigation.

In some examples, eye fluid and waste matter (e.g., emulsified parts of the cataract) are aspirated via hollow needle 16 to a collection receptacle (not shown) by a processor-controlled aspiration pump 26 also comprised in console 28 and using aspiration tubing line 46 running from probe 12 to console 28. In some examples, the same processor that operates the irrigation pump also operates the one or more aspiration pumps, and in some cases, simultaneously. In an example, processor 38 controls an aspiration rate of aspiration pump 26 to maintain IOP (in case of sub-pressure indicated, for example, by sensor 23) within prespecified limits.

As further shown, phacoemulsification probe 12 includes a piezoelectric element such as a piezoelectric element 55 (e.g., one or more piezoelectric crystals) that drives needle 16 to vibrate, for example to vibrate in a resonant vibration mode that is used to break a cataract into small pieces during a phacoemulsification procedure. Console 28 comprises a piezoelectric drive module 30, coupled with the piezoelectric element, using electrical wiring running in cable 33.

Processor 38 further conveys processor-controlled driving signals via cable 33 to, for example, maintain needle 16 at a selected vibration amplitude. The drive module may be realized in hardware or software, for example, in a proportional-integral-derivative (PID) control architecture.

Processor 38 may receive user-based commands via a user interface 40. Examples of user-based commands may include: setting a vibration mode and/or an operating frequency of the piezoelectric element, setting or adjusting an irrigation and/or aspiration rate of the irrigation pump 24 and aspiration pump 26, respectively, setting or adjusting needle 16 stroke amplitude, turning on irrigation and/or aspiration, turning off irrigation and/or aspiration, and/or otherwise controlling the system.

In some examples, the physician uses a foot pedal (not shown) as a means of control. The foot pedal may be operable at a plurality of different positions, each associated with a different type of system activation. Additionally, or alternatively to using a foot pedal, in some systems irrigation is provided (or adjusted, e.g., increased, reduced) in an automated manner, such as in response to activation of aspiration and/or in response to indications obtained by the system sensor(s).

Additionally, or alternatively to the user interface and/or to the foot pedal, processor 38 may receive user-based commands from controls located in a handle 21 of probe 12.

In an example, user interface 40 and display 36 may be integrated into a touch screen graphical user interface. Some or all of the functions of processor 38 may be combined in a single physical component or, alternatively, implemented using multiple physical components. These physical components may comprise hard-wired or programmable devices, or a combination of the two. In some examples, at least some of the functions of processor 38 may be carried out by suitable software stored in a memory 35. This software may be downloaded to a device in electronic form, over a network, for example. Alternatively, or additionally, the software may be stored in tangible, non-transitory computer-readable storage media, such as optical, magnetic, or electronic memory.

The system shown in FIG. 1 may include further elements, which are omitted for clarity of presentation. For example, physician 15 typically performs the procedure using a stereo microscope or magnifying glasses, neither of which are shown. Physician 15 may use other surgical tools in addition to probe 12, which are also not shown in order to maintain clarity and simplicity of presentation.

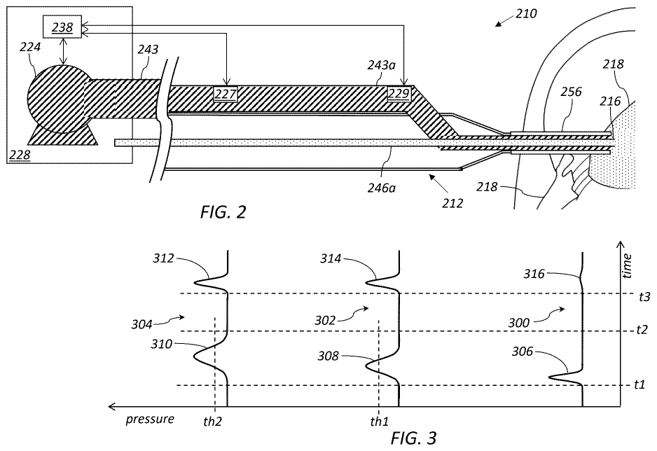

FIG. 2 is a simplified schematic cross-sectional view of exemplary details of a phacoemulsification system 210, according to examples of the present disclosure.

In some examples of the disclosure, FIG. 2 illustrates selected details of system 10 of FIG. 1 where like elements may be indicated with like numerals (e.g., element 243a in FIG. 2 corresponding to element 43 in FIG. 1).

In some examples of the disclosure, system 210 includes a handpiece 212 which hosts an irrigation channel 243a, and an aspiration channel 246a. Where aspiration channel 246a is fluidly connected to a needle 216 at a distal end of handpiece 212 and irrigation channel 243a is fluidly connected to a sleeve 256 at the distal end of handpiece 212. Needle 216 may be coupled with the handpiece 212 and is disposed within sleeve 256, so that irrigation fluid may flow around needle 216. Needle 216 may extend distally beyond a distal end of sleeve 256. Fluid flow through irrigation channel 243a, in some examples is produced by a pump 224 which may be fluidly connected to irrigation channel 243a e.g., distal of handpiece 212. Pump 224 may be hosted externally to handpiece 212 e.g., hosted by a console 228. Although herein termed an “irrigation” channel 243a, in some examples pump 224 is configured to both supply fluid and extract fluid through irrigation channel 243a e.g., extraction provided by pump 224, connected fluidly to irrigation channel by an irrigation tubing 243 (e.g., corresponding to irrigation tubing 43 FIG. 1) reversing a direction of pumping.

In some examples of the disclosure, a cross sectional flow area (area between an inner surface of sleeve 256 and an outer surface of needle 216) of sleeve 256 (cross section taken perpendicular to a long axis of handpiece 212) is smaller than that of the irrigation channel 243a. For example, in some examples, needle 216 may be of any size (e.g., 19-23 gauge) and sleeve 256 may be of any size compatible with the needle gauge (e.g., the sleeve may have a diameter of 1-3 mm. In some examples, irrigation channel 243a has an increasing cross sectional flow area (cross sectional area of fluid within the channel) moving proximally from sleeve 256 towards the proximal end of handpiece 212, and in some examples, the cross sectional flow area may increase from the irrigation channel 243a to the irrigation tube 243 and further to pump 224.

System 210, in some examples, includes a plurality of sensors 227, 229 configured to measure pressure at different locations within irrigation channel 243a. Where the plurality of sensors includes at least a first sensor 229 (also termed “distal sensor”) configured to measure pressure at a first (or distal) location within irrigation channel 243a and a second sensor 227 (also termed “proximal sensor”) configured to measure pressure at a second (or proximal) location within irrigation channel 243a.

Distal sensor 229 may be positioned close to a distal portion (which includes needle 216 and sleeve 256) of handpiece 212.

Distal and proximal sensors 229, 227 may be positioned at a separation of 1-10 cm, or 2-8 cm, or 4-7 cm, or about 5 cm apart from each other e.g., within irrigation channel 243 a.

Sensors 229, 227 may be configured to measure pressure varying between 10-150 mmHg, or 30-100 mmHg, or lower or higher or intermediate pressures or ranges.

Sensors 227, 229 may be coupled with (e.g., via wires or wirelessly) a processor 238. Processor 238 may be hosted by console 228. Processor 238 may generate control signals for control of system pump(s) e.g., irrigation pump 224 (and/or an aspiration pump which is not illustrated in FIG. 2, e.g. aspiration pump 26 shown in FIG. 1).

FIG. 3 is a simplified plot of fluid pressure, with time, at different locations within a phacoemulsification system using a bench top method for simulating surgical events, according to examples of the present disclosure.

FIG. 3, in some examples, illustrates pressure levels 300, 302, 306 at three different locations within or with respect to a phacoemulsification system, where the time scales of the three plots are equivalent. The three plots corresponding to FIG. 2, intraocular pressure 300 within lens capsule 218, pressure 302 within irrigation channel 243a at a location of distal sensor 229, and pressure 304 within irrigation channel 243a at a location of proximal sensor 227.

FIG. 3, in some examples, illustrates two times, t1 and t3 where pressure fluctuation occurs in irrigation channel 243a. The first fluctuation, which occurs after t1, illustrates a fluctuation in IOP 306 and corresponding pressure fluctuation 308, 310 within irrigation channel 243a. Without wanting to be bound by any particular theory, it is theorized that fluctuation in IOP 306 may include a sharp change in pressure (e.g., the fluctuation including high frequencies) which may be diffused by sleeve 256. For example, the dispersion associated with laminar friction between fluid and an external surface of needle 216 as well as that between the fluid and an internal surface of sleeve 256. Where higher frequency portions of the fluctuation in IOP 306 in IOP may be slowed with respect to lower frequency portions of the change in IOP. The diffusion, it is theorized, provides a smeared and/or spread pressure fluctuation 308 at a distal end of irrigation channel 243a and, as the pressure fluctuation passes fluidly through irrigation channel 243a, the pressure fluctuation 310 becomes more smeared and/or spread distally e.g., at a location of proximal sensor 227.

It should be noted that, for slow changes in IOP (e.g., the pressure change signal having little high frequency components) dispersion effects of travel of the signal through the sleeve may be less prominent and may therefore not be identified as IOP changes. However, such changes in IOP, in some examples, are theorized to be less potentially damaging to tissue e.g., not requiring compensation via pump control.

The second fluctuation, which occurs after t3, illustrates a pressure fluctuation 312, 314 in irrigation fluid pressure associated with pressure changes within irrigation channel, not originating at the eye. For example, pressure fluctuation(s) associated with operation of pump 224. Without wanting to be bound by any theory it is theorized that, such pressure fluctuation 312, 314 will be experienced throughout irrigation channel 243 a within a same time frame (e.g., within 1 second, or within 0.5 seconds, or within lower, or higher, or intermediate time durations or ranges) without significant spreading of the fluctuation. The theory being that irrigation channel 243a is, in some examples, sufficiently large with respect to wavelengths of the pressure fluctuation signal and with respect to laminar friction at the walls of irrigation channel 243a does not disrupt fluid flow significantly.

In some examples, pressure fluctuation 312, 314 in irrigation channel 243a is not transferred (or is transferred minimally as illustrated by fluctuation in IOP 316) to fluid of sleeve 216, as, for example, narrowing of the fluid cross section from irrigation channel 243a of handpiece 212 to sleeve 256, which acts as a low-pass filter and/or damper for pressure fluctuation 312, 314 in irrigation channel 243a.

Although not illustrated in FIG. 3, pressure fluctuations may be attenuated by their passage through the sleeve and/or irrigation channel. In some examples, attenuation of pressure signals travelling through the irrigation channel is compensated for.

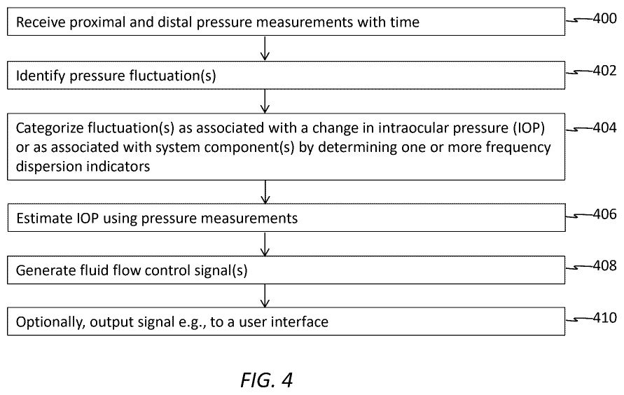

FIG. 4 is a method of controlling a phacoemulsification system, according to examples of the present disclosure.

At step 400, proximal (second) and distal (first) pressure measurement signals based on time from within an irrigation channel are received, for example, from within irrigation channel 43a of FIG. 1 or irrigation channel of 243a FIG. 2. The proximal and distal pressure measurement signals may be received from and/or provided by proximal sensor 227 and distal sensor 229 shown in FIG. 2, respectively. The pressure measurement signals may include continuous or periodic measurements of pressure. Periodic/discrete measurements may be carried out rapidly with respect to expected fluctuation(s) in IOP. In some examples, the sensors provide digital outputs of the measurements.

Optionally, the pressure drop along the irrigation channel, which may be associated with flow of fluid through the irrigation channel and laminar friction (e.g. at the walls of the irrigation channel) may be compensated for in the pressure measurement signals. The compensation is based on an assumption of laminar flow (e.g., according to Bernoulli's principle).

At step 402, pressure fluctuation(s) are identified within the pressure measurement signals. For example, where one or more time periods are identified (e.g., data associated with time period(s)) where one or both of the proximal and distal pressure measurement signals deviates by more than a threshold. In some examples, a pressure fluctuation is only identified when both of the proximal and distal pressure measurement signals deviate by a respective threshold.

In an example, to identify a pressure fluctuation, the proximal pressure measurement may be compared with a first threshold and the distal pressure measurement signal may be compared with a second threshold, where the first and second thresholds may be different.

Measurement signal vector sets may then be outputted for each identified pressure fluctuation, each measurement signal vector set including a measurement signal vector for each pressure measurement signal. A measurement signal vector includes pressure measurement signal data for a time period in which the pressure measurement fluctuation has been identified, and the signal data including sequential pressure measurement signal values based on time. The measurement signal vector set includes sequential pressure measurement signal values based on time for each of the pressure measurement signals. In some examples, measurement signal vectors include pressure measurement signal values surrounding (in time) data points where the value(s) deviate by a threshold, for example, to provide pressure data of a whole fluctuation signal (e.g., including tail ends of the fluctuation which may include relevant frequency data).

For example, referring to FIG. 3, a pressure fluctuation may be identified as pressure measurement signal 302 being above a first threshold th1 during fluctuation 308 and/or as pressure measurement signal 304 being above a second threshold th2 during fluctuation 308. The pressure measurement vector set, for fluctuation 310, 308 may include data values of the pressure measurement signals for time extending from t1 to t2, although not all pressure measurement signals are above the respective threshold for this entire time duration. A potential advantage being more accurate frequency dispersion indicator(s) at step 404 as lower intensity portions (below threshold) of the pressure measurement signal may include frequency information, which otherwise would not be considered.

At step 404, the pressure fluctuation(s) (e.g., measurement vector sets as described in step 402) are characterized by determining/calculating one or more frequency dispersion indicators (e.g., for each time period in which a pressure fluctuation is identified in step 402). The one or more frequency dispersion indicators are used to characterize the pressure fluctuation as associated with a change in IOP or as associated with other system component(s) and/or pressure fluctuation(s), e.g., pressure fluctuation associated with the irrigation pump.

In some examples, the one or more frequency dispersion indicators include a measure of correlation between the measurement signals (e.g., correlation as a multiplication of two signals by their corresponding samples, normalized by a norm of each of the measurement signal vectors). In some examples, the correlation may be compared with a threshold, and those pressure fluctuation(s) which are above the threshold are characterized as being associated with IOP.

Additionally or alternatively, in some examples, the one or more frequency dispersion indicators include a lag between the proximal and distal measurement signals. Where the lag may be determined by calculating a cross correlation for the measurement signal vectors. Correlation is calculated with a temporal shift, until a peak in correlation is determined, the lag being the temporal shift associated with the peak correlation. In some examples, the lag may be compared with a threshold, and those pressure fluctuation(s) which exceed the threshold are characterized as being associated with IOP.

Alternatively or additionally, in some examples, the one or more frequency dispersion indicators may be determined from a phase differential factor. The phase differential factor is determined by decomposing the pressure measurement vectors to the frequency domain, e.g., using Fourier transforms, to provide a set of complex numbers for each pressure measurement vector. Each complex number is indicative of the pressure measurement at a given frequency or frequency range. In some examples, phase of the complex numbers is determined, and corresponding phases for the two pressure measurement signals (e.g., phases associated with a same frequency) are subtracted from each other to give the phase differential factor, which is an indication of relative delay for each frequency (or frequency range). A phase difference that varies with frequency (e.g., as described hereinabove), may indicate dispersion; different frequencies experiencing different delays.

A potential benefit of using the phase differential factor to characterize frequency fluctuation(s) is that bias compensation for a bias associated with pressure drop along the irrigation channel may not be required.

In some examples, a frequency dispersion indicator may include one or more of a slope of the phase differential factor, a weighted average of the phase differential factor, or a phase difference ratio determined from the phase differential factor.

For example, if the slope of the phase differential factor varies with frequency (e.g., is not constant), the pressure measurement signals may be characterized as exhibiting sufficient dispersion to characterize the frequency fluctuation as being associated with IOP.

In some examples, a frequency dispersion indicator may include a weighted average of the phase difference, giving more weight to higher frequencies, which is then compared with a threshold. When exceeding the threshold, the pressure measurement signals may be characterized as exhibiting sufficient dispersion to characterize the frequency fluctuation as being associated with IOP.

In some examples, a frequency dispersion indicator may include a phase difference ratio, where the ratio may be of high vs. low frequencies. This ratio, in some examples, may be compared to a threshold and when exceeding the threshold, the pressure measurement signals may be characterized as exhibiting sufficient dispersion to characterize the frequency fluctuation as being associated with IOP.

Additionally or alternatively, in some examples, the one or more frequency dispersion indicators may include a comparison of frequency-specific cross-correlations. Signal measurement vectors may be decomposed into frequency bands (e.g., via filtering or wavelet transform), and then the cross-correlation lag for each band is determined separately. If higher frequency bands exhibit larger lags than lower frequency bands, this may indicate dispersion. For example, the lag differences between high and low frequency bands may be compared with a threshold to characterize the pressure measurement signals as exhibiting sufficient dispersion to characterize the frequency fluctuation as being associated with IOP.

At step 406, the first (distal) pressure measurement signal is used to estimate the IOP, e.g., for those pressure fluctuation(s) characterized as being associated with IOP fluctuation(s).

At step 408, fluid flow is controlled, e.g., using the indication of IOP as calculated at step 406. Where, in some examples, control signal(s) are generated using an error signal. Where the error signal is determined as a difference between a desired IOP and the indication of IOP as calculated at step 406. In some examples, the desired IOP is set by a physician (e.g., via user interface 40). In some examples, control signal(s) are generated by smoothing and/or processing the error signal. The control signal may be generated using or be an output of a feedback-control loop (e.g., a PID proportional-integral-derivative control architecture) to which the error signal is an input. Referring to FIG. 1 and FIG. 2, processor 38, 238 conveys processor-controlled pump driving signals to pump 24, 224, to maintain IOP within an allowed/selected range.

The control signal(s) may be configured to adjust one or more features of pump operation including one or more of activation/deactivation of the pump, direction of the pump, speed of the pump (e.g., volume/time), and pressure of pumping.

Control may include controlling actuator(s) of the irrigation pump (e.g., irrigation pump 224 of FIG. 2) to increase or decrease fluid supply through the irrigation channel 243a and/or reverse operation of the pump to extract fluid through the sleeve (e.g., sleeve 256 of FIG. 2). Where control may additionally or alternatively include controlling actuator(s) of the aspiration pump (e.g., via one or more control signal(s) generated by the controller and conveyed to the aspiration pump).

At optional step 410, a user (e.g., medical practitioner performing the phacoemulsification procedure) is informed of a change in control or change in IOP. For example, where a signal may be outputted to a user interface. In some examples, the signal is an indication that a change in IOP has occurred and/or a determined IOP is outputted. Optionally, the outputted signal is saved to a memory.

In some examples, change(s) in IOP are evaluated and one or more output may be provided based on the evaluation. For example, fluctuations outside a normal range of expected fluctuations may trigger a warning output, e.g., if pressure change is above or below a normal and/or acceptable range.

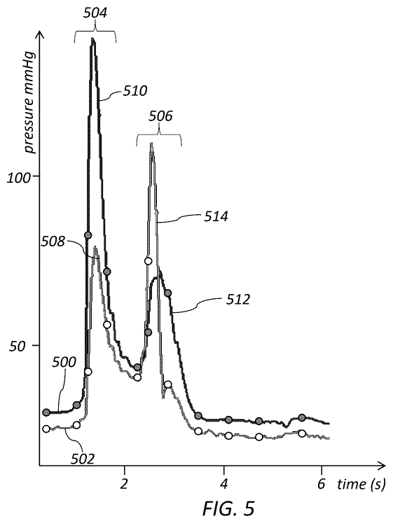

FIG. 5 is a plot of pressure measurement signals versus time, according to examples of the present disclosure.

FIG. 5 illustrates exemplary measurements within an irrigation channel where plot 500 is a measurement signal from a proximal sensor (e.g., proximal sensor 227 of FIG. 2) and plot 502 is a measurement signal from a distal sensor (e.g., distal sensor 229 of FIG. 2).

The plots illustrate two sets of peaks 504, 506, where a first peak 504 demonstrates pressure fluctuation having a source outside the eye. First peak 504 was provided by injecting additional fluid into the irrigation channel. As theorized, both a corresponding peak in pressure measured distally 508 and proximally 510 occur within the same time frame, although distal peak 508 has reduced amplitude, e.g., associated with flow of fluid through the irrigation channel.

A second peak 506 demonstrates pressure fluctuation associated with fluctuation in IOP. Second peak 506 was provided by brief manual application of pressure to the cornea. As theorized, a corresponding peak 512 in pressure measured proximally is delayed and spread in comparison to a corresponding peak 514 measured distally indicating frequency dispersion of the change in IOP as the pressure fluctuation travels proximally through the irrigation channel.

### EXAMPLES

Following is a non-exclusive list of some exemplary examples of the disclosure. The present disclosure also includes examples which include fewer than all the features in an example and examples using features from multiple examples, even if not listed below.

Example 1. A surgical system (10, 210), comprising: a pump (224) configured to transmit and/or evacuate fluid; a handpiece (12, 212) comprising: a distal portion configured to be inserted into a patient's eye comprising a needle (16, 216) and a sleeve (56, 256); an irrigation channel (43a, 243a) fluidly coupled with said pump (224) and forming a fluid path from a proximal end of the handpiece to said sleeve (56, 256); a first sensor (229) and a second sensor (227) configured to sense pressure at separate locations along said fluid path within said irrigation channel (43a, 243a); and circuitry (38, 238) configured to: receive a first pressure measurement signal (302) from said first sensor (229) and a second pressure measurement signal (304) from said second sensor (227); identify one or more pressure fluctuations in said first pressure measurement signal and/or said second pressure measurement signal; compare said first pressure measurement signal and said second pressure measurement signal to determine one or more frequency dispersion indicators; characterize said one or more pressure fluctuations based on said one or more frequency dispersion indicators as being associated with an intraocular pressure (IOP) change or as associated with a system component; and for a pressure fluctuation associated with said IOP change: determine an estimate of IOP from said first pressure measurement signal; and control said pump (224) based on said estimate of IOP.

Example 2. The surgical system according to Example 1, wherein said circuitry (38, 238) is configured to one or more of: identify each of said one or more pressure fluctuations as a concurrent time portion of said first pressure measurement signal and said second pressure measurement signal; and characterize said one or more pressure fluctuations by comparing each of said one or more frequency dispersion indicators with a corresponding threshold.

Example 3. The surgical system according to any one of Examples 1-2, wherein said one or more frequency dispersion indicators includes a lag between said first pressure measurement signal and said second pressure measurement signal; and said circuitry is configured to characterize each said pressure fluctuation as associated with IOP if a lag associated with the pressure fluctuation exceeds a threshold.

Example 4. The surgical system according to Example 3, wherein said circuitry (38, 238) is configured to determine said lag by determining a cross-correlation between said first and said second pressure measurement signal.

Example 5. The surgical system according to any one of Examples 1-4, wherein said circuitry (38, 238) is configured to compare said first pressure measurement signal and said second pressure measurement signal in a frequency domain to provide a phase differential factor, wherein said one or more frequency dispersion indicators includes a parameter determined from said phase differential factor.

Example 6. The surgical system according to Example 5, wherein said one or more frequency dispersion indicators include one or more of; a slope of said phase differential factor; a weighted average of said phase differential factor; and a phase difference ratio determined from said phase differential factor.

Example 7. The surgical system according to any one of Examples 1-6, wherein said circuitry (38, 238) is configured to control said pump (224) by generating one or more control signal.

Example 8. The surgical system according to Example 7, wherein said one or more of: said generating comprises determining an error signal as a difference between said estimate of IOP and a desired IOP; and said generating comprises providing said error signal to a closed looped control architecture, wherein said one or more control signal is an output of said control architecture.

Example 9. The surgical system according to Example 8, wherein said closed loop control includes a proportional-integral-derivative (PID) control architecture.

Example 10. The surgical system according to any one of Examples 8-9, wherein said one or more control signal includes a pump control signal for control of said pump (224); and wherein said pump control signal is configured to instruct said pump (224) to change one or more of: activation state of said pump (224), direction of fluid flow from the pump (224), speed of pumping, and pressure of pump fluid.

Example 11. The surgical system according to any one of Examples 8-9, wherein said system (10, 210) comprises a user interface (36, 40) and, said control signal is configured to control output at said user interface (36, 40) based on said error signal.

Example 12. The surgical system according to any one of Examples 1-11, wherein said irrigation channel has a cross-sectional flow area which is larger than a cross-sectional flow area of said sleeve; and wherein said handpiece (12, 212) comprises a needle (16, 216) extending through said sleeve (56, 256), said flow area of said sleeve (56, 256) formed by an area between an inner surface of a sleeve wall and an outer surface of said needle (16, 216).

Example 13. The surgical system according to Example 12, comprising an aspiration pump (26) fluidly connected to said needle (16, 216); wherein said handpiece (12, 212) comprises an aspiration channel (46a, 246a) forming an aspiration fluid path from said needle to said aspiration pump, the aspiration pump configured to evacuate material along said aspiration fluid path.

Example 14. The surgical system according to any one of Examples 1-13, wherein said first sensor (229) is located distally of said second sensor (227).

Example 15. A method of operating a phacoemulsification device, comprising: providing a surgical system (10, 210) comprising a pump (24, 224) and a handpiece (12, 212), wherein the handpiece (12, 212) comprises an irrigation channel (43a, 243a), a needle (16, 216) and a sleeve (56, 256) coaxially surrounding at least a portion of the needle, (16, 216) wherein the irrigation channel (43a, 243a) is fluidly coupled with the sleeve (56, 256), and wherein the sleeve (16, 216) comprises a cross-sectional flow area between an outer surface of the needle (16, 216) and the inner surface of the sleeve (16, 216) that is smaller than a cross-sectional area of the irrigation channel; receiving a first pressure measurement signal indicative of pressure at a first location in said irrigation channel and a second pressure measurement signal indicative of pressure at a second location in said irrigation channel; identifying pressure fluctuations occurring at one or both said first pressure measurement signal and said second pressure measurement signal; comparing said first pressure measurement signal and said second pressure measurement signal, for said pressure fluctuations, to provide one or more frequency dispersion indicators indicative of frequency dispersion between said first pressure measurement signal and said second pressure measurement signal; characterizing said pressure fluctuations as associated with intraocular pressure (IOP) or as associated with system pressure, based on said one or more frequency dispersion indicators; and for a pressure fluctuation associated with IOP: determining an estimate of IOP from said first pressure measurement signal; and controlling said pump based on said estimate of IOP.

Example 16. A surgical system, comprising: a pump configured to transmit and/or evacuate fluid; a handpiece comprising: a distal portion configured to be inserted into a patient's eye comprising a needle and a sleeve; an irrigation channel fluidly coupled with said pump and forming a fluid path from a proximal end of the handpiece to said sleeve; a first sensor and a second sensor configured to sense pressure at separate locations along said fluid path within said irrigation channel, where said first sensor is located distally of said second sensor; and circuitry configured to: receive a first pressure measurement signal from said first sensor and a second pressure measurement signal from said second sensor; identify one or more pressure fluctuations in said first pressure measurement signal and/or said second pressure measurement signal; compare said first pressure measurement signal and said second pressure measurement signal to determine one or more frequency dispersion indicators; characterize said one or more pressure fluctuations based on said one or more frequency dispersion indicators as being associated with an intraocular pressure (IOP) change or as associated with a system component; and for a pressure fluctuation associated with said IOP change: determine an estimate of IOP from said first pressure measurement signal; and control said pump based on said estimate of IOP.

Example 17. The surgical system according to Example 16, wherein said circuitry is configured to one or more of: identify each of said one or more pressure fluctuations as a concurrent time portion of said first pressure measurement signal and said second pressure measurement signal; and characterize said one or more pressure fluctuations by comparing each of said one or more frequency dispersion indicators with a corresponding threshold.

Example 18. The surgical system according to any one of Examples 16-17, wherein said one or more frequency dispersion indicators includes a lag between said first pressure measurement signal and said second pressure measurement signal; and said circuitry is configured to characterize each said pressure fluctuation as associated with IOP if a lag associated with the pressure fluctuation exceeds a threshold.

Example 19. The surgical system according to Example 18, wherein said circuitry is configured to determine said lag by determining a cross-correlation between said first and said second pressure measurement signal.

Example 20. The surgical system according to any one of Examples 16-17, wherein said circuitry is configured to compare said first pressure measurement signal and said second pressure measurement signal in a frequency domain to provide a phase differential factor, wherein said one or more frequency dispersion indicators includes a parameter determined from said phase differential factor including one or more of; a slope of said phase differential factor; a weighted average of said phase differential factor; and a phase difference ratio determined from said phase differential factor.

Example 21. The surgical system according to any one of Examples 16-17, wherein said circuitry (38, 238) is configured to control said pump by generating one or more control signal.

Example 22. The surgical system according to Example 21, wherein said one or more of: said generating comprises determining an error signal as a difference between said estimate of IOP and a desired IOP; and said generating comprises providing said error signal to a closed looped control architecture, wherein said one or more control signal is an output of said control architecture.

Example 23. The surgical system according to any one of Examples 22, wherein said one or more control signal includes a pump control signal for control of said pump; and wherein said pump control signal is configured to instruct said pump to change one or more of: activation state of said pump, direction of fluid flow from the pump, speed of pumping, and pressure of pump fluid.

Example 24. The surgical system according to any one of Examples 16-17, wherein said irrigation channel has a cross-sectional flow area which is larger than a cross-sectional flow area of said sleeve; wherein said handpiece comprises a needle extending through said sleeve, said flow area of said sleeve formed by an area between an inner surface of a sleeve wall and an outer surface of said needle; and an aspiration pump fluidly connected to said needle; wherein said handpiece comprises an aspiration channel forming an aspiration fluid path from said needle to said aspiration pump, the aspiration pump configured to evacuate material along said aspiration fluid path.

Example 25. A method of operating a phacoemulsification device, comprising: providing a surgical system comprising a pump and a handpiece, wherein the handpiece comprises an irrigation channel, a needle and a sleeve coaxially surrounding at least a portion of the needle, wherein the irrigation channel is fluidly coupled with the sleeve, and wherein the sleeve comprises a cross-sectional flow area between an outer surface of the needle and the inner surface of the sleeve that is smaller than a cross-sectional area of the irrigation channel; receiving a first pressure measurement signal indicative of pressure at a first location in said irrigation channel and a second pressure measurement signal indicative of pressure at a second location in said irrigation channel; identifying pressure fluctuations occurring at one or both said first pressure measurement signal and said second pressure measurement signal; comparing said first pressure measurement signal and said second pressure measurement signal, for said pressure fluctuations, to provide one or more frequency dispersion indicators indicative of frequency dispersion between said first pressure measurement signal and said second pressure measurement signal; characterizing said pressure fluctuations as associated with intraocular pressure (IOP) or as associated with system pressure, based on said one or more frequency dispersion indicators; and for a pressure fluctuation associated with IOP: determining an estimate of IOP from said first pressure measurement signal; and controlling said pump based on said estimate of IOP.

Those skilled in the art to which the present disclosure pertains, can appreciate that while the present disclosure has been described in terms of preferred examples, the concept upon which this disclosure is based may readily be utilized as a basis for the designing of other structures, systems and processes for carrying out the several purposes of the present disclosure.

Also, it is to be understood that the phraseology and terminology employed herein are for the purpose of description and should not be regarded as limiting. It should be noted that the words “comprising”, “including” and “having” as used throughout the appended claims are to be interpreted to mean “including but not limited to”. The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases, and disjunctively present in other cases. The term “each” may not be exclusively understood as referring to each and every, and when technically relevant may also refer to “at least some”.

All patents and patent applications mentioned in this specification are herein incorporated in their entirety by reference into the specification, to the same extent as if each individual patent or patent application was specifically and individually indicated to be incorporated herein by reference. In addition, citation or identification of any reference in this application shall not be construed as an admission that such reference is available as prior art to the present disclosure.

It is important, therefore, that the scope of the present disclosure is not construed as being limited by the illustrative examples set forth herein. Other variations are possible within the scope of the present disclosure as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

1. A surgical system, comprising:
a pump configured to transmit and/or evacuate fluid;
a handpiece comprising:
a distal portion configured to be inserted into a patient's eye comprising a needle and a sleeve;
an irrigation channel fluidly coupled with said pump and forming a fluid path from a proximal end of the handpiece to said sleeve;
a first sensor and a second sensor configured to sense pressure at separate locations along said fluid path within said irrigation channel; and
circuitry configured to:
receive a first pressure measurement signal from said first sensor and a second pressure measurement signal from said second sensor;
identify one or more pressure fluctuations in said first pressure measurement signal and/or said second pressure measurement signal;
compare said first pressure measurement signal and said second pressure measurement signal to determine one or more frequency dispersion indicators;
characterize said one or more pressure fluctuations based on said one or more frequency dispersion indicators as being associated with an intraocular pressure (IOP) change or as associated with a system component; and
for a pressure fluctuation associated with said IOP change:
determine an estimate of IOP from said first pressure measurement signal; and
control said pump based on said estimate of IOP.

2. The surgical system according to claim 1, wherein said circuitry is configured to identify each of said one or more pressure fluctuations as a concurrent time portion of said first pressure measurement signal and said second pressure measurement signal.

3. The surgical system according to claim 1, wherein said circuitry is configured to characterize said one or more pressure fluctuations by comparing each of said one or more frequency dispersion indicators with a corresponding threshold.

4. The surgical system according to claim 3, wherein said one or more frequency dispersion indicators includes a lag between said first pressure measurement signal and said second pressure measurement signal; and
said circuitry is configured to characterize each said pressure fluctuation as associated with IOP if a lag associated with the pressure fluctuation exceeds a threshold.

5. The surgical system according to claim 4, wherein said circuitry is configured to determine said lag by determining a cross-correlation between said first and said second pressure measurement signal.

6. The surgical system according to claim 1, wherein said circuitry is configured to compare said first pressure measurement signal and said second pressure measurement signal in a frequency domain to provide a phase differential factor, wherein said one or more frequency dispersion indicators includes a parameter determined from said phase differential factor.

7. The surgical system according to claim 6, wherein said one or more frequency dispersion indicators include one or more of;
a slope of said phase differential factor;
a weighted average of said phase differential factor; and
a phase difference ratio determined from said phase differential factor.

8. The surgical system according to claim 1, wherein said circuitry is configured to control said pump by generating one or more control signal.

9. The surgical system according to claim 8, wherein said generating comprises determining an error signal as a difference between said estimate of IOP and a desired IOP.

10. The surgical system according to claim 9, wherein said generating comprises providing said error signal to a closed looped control architecture, wherein said one or more control signal is an output of said control architecture.

11. The surgical system according to claim 10, wherein said closed loop control includes a proportional-integral-derivative (PID) control architecture.

12. The surgical system according to claim 8, wherein said one or more control signal includes a pump control signal for control of said pump;
wherein said pump control signal is configured to instruct said pump to change one or more of: activation state of said pump, direction of fluid flow from the pump, speed of pumping, and pressure of pump fluid.

13. The surgical system according to claim 10, wherein said system comprises a user interface and, said control signal is configured to control output at said user interface based on said error signal.

14. The surgical system according to claim 1, wherein said irrigation channel has a cross-sectional flow area which is larger than a cross-sectional flow area of said sleeve.

15. The surgical system according to claim 14, wherein said handpiece comprises a needle extending through said sleeve, and wherein said flow area of said sleeve is formed by an area between an inner surface of a sleeve wall and an outer surface of said needle.

16. The surgical system according to claim 15, comprising an aspiration pump fluidly coupled with said needle;
wherein said handpiece comprises an aspiration channel forming an aspiration fluid path from said needle to said aspiration pump, the aspiration pump configured to evacuate material along said aspiration fluid path.

17. The surgical system according to claim 1, wherein said first sensor is located distally of said second sensor.

18. A method of operating a phacoemulsification device, comprising:
providing a surgical system comprising a pump and a handpiece, wherein the handpiece comprises an irrigation channel, a needle and a sleeve coaxially surrounding at least a portion of the needle, wherein the irrigation channel is fluidly coupled with the sleeve, and wherein the sleeve comprises a cross-sectional flow area between an outer surface of the needle and the inner surface of the sleeve that is smaller than a cross-sectional area of the irrigation channel;
receiving a first pressure measurement signal indicative of pressure at a first location in said irrigation channel and a second pressure measurement signal indicative of pressure at a second location in said irrigation channel;
identifying pressure fluctuations occurring at one or both said first pressure measurement signal and said second pressure measurement signal;
comparing said first pressure measurement signal and said second pressure measurement signal, for said pressure fluctuations, to provide one or more frequency dispersion indicators indicative of frequency dispersion between said first pressure measurement signal and said second pressure measurement signal;
characterizing said pressure fluctuations as associated with intraocular pressure (IOP) or as associated with system pressure, based on said one or more frequency dispersion indicators; and
for a pressure fluctuation associated with IOP:
determining an estimate of IOP from said first pressure measurement signal; and
controlling said pump based on said estimate of IOP.

19. The method according to claim 18, wherein one or more of:
said identifying comprises identifying each of said pressure fluctuations as a concurrent time portion of said first pressure measurement signal and said second pressure measurement signal; and
said characterizing comprises comparing each of said one or more frequency dispersion indicators with a corresponding threshold.

20. The method according to claim 18, wherein said one or more frequency dispersion indicators includes one or more of:
a lag between said first pressure measurement signal and said second pressure measurement signal;
a slope of a phase differential factor;
a weighted average of a phase differential factor; and
a phase difference ratio determined from a phase differential factor.
